(19) United States
(12) Patent Application Publication
(10) Pub. No.: US 2007/0088079 A1
(43) Pub. Date: Apr. 19, 2007

(54) Prophylactic or therapeutic agent for sleep disorder

(75) Inventors: Keisuke Hirai, Osaka-shi (JP); Masaomi Miyamoto, Osaka-shi (JP)

Correspondence Address: EDWARDS & ANGELL, LLP, P.O. BOX 55874, BOSTON, MA, 02205, US

(73) Assignee: Takeda Pharmaceutical Company Limited, Osaka (JP)

(21) Appl. No.: 11/524,715

(22) Filed: Sep. 20, 2006

Related U.S. Application Data
(60) Provisional application No. 60/818,213, filed on Jun. 30, 2006.

(30) Foreign Application Priority Data
Sep. 20, 2005 (JP) 2005-272260
Apr. 21, 2006 (JP) 2006-117524

(51) Int. Cl. A61K 31/343

(52) U.S. Cl. 514/468

(57) ABSTRACT

By combining one or more drugs selected from the group consisting of sedative antidepressants and antihistamines with (S)—N-[2-(1,6,7,8-tetrahydro-2H-indeno[5,4-b]furan-8-yl)ethyl]propionamide, there is provided a prophylactic or therapeutic agent for sleep disorder that induces natural sleep, shortens sleep latency, increases deep sleep, is excellent in maintaining sleep, and exerts to attain appropriate sleep duration.

### TECHNICAL FIELD

The present invention relates to a prophylactic or therapeutic agent for sleep disorder.

### BACKGROUND ART

(S)—N-[2-(1,6,7,8-Tetrahydro-2H-indeno[5,4-b]furan-8-yl)ethyl]propionamide (general name: Ramelteon (hereinafter, sometimes referred to as compound A)) disclosed in W097/32871 is a compound having an excellent melatonin agonistic action and expected as an extremely advantageous prophylactic or therapeutic agent for sleep disorder that induces natural sleep.

The prophylactic or therapeutic agents for sleep disorder is desired to have properties of, for example, inducing natural sleep, having short sleep latency, increasing deep sleep (i.e., prolonging slow-wave sleep (SWS) duration), maintaining sleep and achieving appropriate sleep duration.

Although benzodiazepine or non-benzodiazepine drugs acting on GABA-A receptor are mainly used as prophylactic or therapeutic agents for sleep disorder, sedative antidepressants are sometimes used for insomnia etc. (for example, Roehrs T., et al., Sleep Med. 5(5):463-6, September 2004). On the other hand, antihistamines are also sometimes used as over-the-counter (OTC) drugs for mild insomnia. These drugs have characteristics of increasing deep sleep, but when used alone, their drug efficacy is insufficient as therapeutic agents for sleep disorder.

### DISCLOSURE OF THE INVENTION

Problems to be Solved by the Invention

The above-mentioned compound A, even when used alone, is expected to satisfy these needs, but there is a need for development of drugs satisfying these needs at higher degrees.

Accordingly, the object of the present invention is to provide a prophylactic or therapeutic agent for sleep disorder that induces natural sleep, shortens sleep latency, increases deep sleep, is excellent in maintaining sleep, and exerts to attain appropriate sleep duration.

Means of Solving the Problems As a result of intensive studies, the present inventors found out that sleep latency is shortened, deep sleep is increased, maintenance of sleep is excellent, and appropriate sleep duration can be attained by using an sedative antidepressant and/or an antihistamine which has a feature of increasing deep sleep, in combination with compound A which is an extremely advantageous prophylactic or therapeutic agent for sleep disorder inducing natural sleep (as a combination preparation, a blended preparation or a concomitant preparation), and the present invention has been completed.

That is, the present invention provides:
[1] A pharmaceutical composition for preventing or treating sleep disorder, which comprises (S)—N-[2-(1,6,7,8-tetrahydro-2H-indeno[5,4-b]furan-8-yl)ethyl]propionamide in combination with one or more drugs selected from the group consisting of sedative antidepressants and antihistamines;
[2] A pharmaceutical composition for preventing or treating sleep disorder, which comprises (S)—N-[2-(1,6,7,8-tetrahydro-2H-indeno[5,4-b]furan-8-yl)ethyl]propionamide in combination with doxepin;
[3] Use of (S)—N-[2-(1,6,7,8-tetrahydro-2H-indeno[5,4-b]furan-8-yl)ethyl]propionamide in combination with one or more drugs selected from the group consisting of sedative antidepressants and antihistamines, for the manufacture of a pharmaceutical composition for preventing or treating sleep disorder;
[4] Use of (S)—N-[2-(1,6,7,8-tetrahydro-2H-indeno[5,4-b]furan-8-yl)ethyl]propionamide in combination with doxepin, for the manufacture of a pharmaceutical composition for preventing or treating sleep disorder;
[5] A method for preventing or treating sleep disorder, which comprises administering (S)—N-[2-(1,6,7,8-tetrahydro-2H-indeno[5,4-b]furan-8-yl)ethyl]propionamide in combination with one or more drugs selected from the group consisting of sedative antidepressants and antihistamines to a subject; and
[6] A method for preventing or treating sleep disorder, which comprises administering (S)—N-[2-(1,6,7,8-tetrahydro-2H-indeno[5,4-b]furan-8-yl)ethyl]propionamide in combination with doxepin to a subject.

### BRIEF DESCRIPTION OF DRAWINGS

FIG. 1 is a graph showing REM time, SWS time, and total sleep duration.

FIG. 2 is a graph showing a power spectrum during SWS in a single administration group of compound A.

FIG. 3 is a graph showing a power spectrum during SWS in a single administration group of doxepin.

FIG. 4 is a graph showing a power spectrum during SWS in a combined administration group of compound A and doxepin.

### BEST MODE FOR CARRYING OUT THE INVENTION

Specific examples of the sedative antidepressant used in the present invention include doxepin, trazodone, nefasodone, paroxetine, amitriptyline, maprotiline, mianserin, fluvoxamine, trimipramine, setiptiline, desipramine, imipramine and mirtazapine (Org-4420 (S-mirtazapine)). Preferably, the sedative antidepressant used in the present invention is, for example, a tricyclic antidepressant (e.g., doxepin, amitriptyline, trimipramine, desipramine and imipramine). As the tricyclic antidepressant, preferred is a compound represented by the formula: 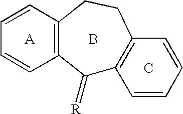
wherein rings A and C each represent a 6-membered aromatic ring, ring B represents a 7-membered carbocyclic ring or a 7-membered heterocyclic ring having one or more hetero atoms selected from nitrogen atom, oxygen atom and sulfur atom as ring constituent atoms, R represents a C₁₋₄ alkyl group substituted with dimethylamino group or a C₁₋₄ alkylidene group substituted with dimethylamino group, and [symbol] represents a single bond or a double bond, or a salt thereof.

The sedative antidepressants illustrated herein are a known compound, and each of them is available as commercial products or can be easily synthesized according to known methods described in Stach K. et al., Monatsh. Chem., 1967, 93, 896; U.S. Pat. No. 3,354,155; Miodownik, A. et al., Synth. Commun., 1981, 11, 241; etc.

Specific examples of the antihistamines used in the present invention include diphenhydramine, doxylamine, azatadine, brompheniramine, cetirazine, chlorpheniramine, clemastine, cyproheptadine, desloratadine, dexchlorpheniramine, dimenhydrinate, fexofenadine, hydroxyzine, loratadine and phenindamine. Preferably, the antihistamine used in the present invention is, for example, a histamine H1 antagonist.

The antihistamines exemplified herein are a known compound, and each of them is available as commercial products, or can be easily synthesized according to methods known in the art.

These sedative antidepressants and/or antihistamines may be used alone or in combination of two or more thereof. Preferred sedative antidepressants and/or antihistamines are those having both properties of a tricyclic antidepressant and a histamine H1 antagonist. In particular, doxepin is preferred.

The compound A used in the present invention can be produced according to methods described in, for example, WO97/32871 or analogous method thereto.

In the present specification, compounds or drugs may be a free form or a salt, or any one of non-hydrate or hydrate, or may be any one of a racemate or an optically active compound, unless otherwise stated. Such salt is not particularly limited insofar as it is a pharmacologically acceptable salt, and examples thereof include salts with inorganic bases, salts with organic bases, salts with inorganic acids, salts with organic acids, and salts with basic or acidic amino acids. Preferable examples of the salts with inorganic bases include, for example, alkali metal salts such as sodium salt and potassium salt, alkaline earth metal salts such as calcium salt and magnesium salt, aluminum salt, ammonium salt, and the like. Preferable examples of the salts with organic bases include, for example, salts with trimethyl amine, triethyl amine, pyridine, picoline, 2,6-lutidine, ethanolamine, diethanolamine, triethanolamine, cyclohexylamine, dicyclohexylamine, N,N′-dibenzylethylenediamine, and the like. Preferable examples of the salts with inorganic acids include, for example, salts with hydrochloric acid, hydrobromic acid, nitric acid, sulfuric acid, phosphoric acid, and the like. Preferable examples of the salts with organic acids include, for example, salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid, and the like. Preferable examples of the salts with basic amino acids include, for example, salts with arginine, lysine, ornithine, and the like. Preferable examples of the salts with acidic amino acids include, for example, salts with aspartic acid, glutamic acid, and the like.

In the prophylactic or therapeutic agent for sleep disorder of the present invention, all of the active ingredients may be contained in one preparation, or each or a part of the active ingredients may be compounded in separate preparations. That is, the prophylactic or therapeutic agent for sleep disorder of the present invention may be, for example, (1) a single preparation wherein active ingredients, i.e., one or more drugs selected from the group consisting of sedative antidepressants and antihistamines, and compound A are formulated with an appropriate pharmaceutically acceptable excipient etc., if desired, according to a known production method for formulating pharmaceutical preparations, (2) preparations wherein each of the active ingredients, i.e., one or more drugs selected from the group consisting of sedative antidepressants and antihistamines, and compound A is formulated with an appropriate pharmaceutically acceptable excipient etc., if desired, so as to be used in combination (combined use) simultaneously or at different times, or (3) a combined set (kit products etc.) of preparations wherein each of the active ingredients is separately formulated appropriately with an excipient by a conventional method.

Dosage forms of the prophylactic or therapeutic agent for sleep disorder of the present invention are not particularly limited, and preferred is a dosage form which can be administered orally to a patient (for example, tablets, fine granules, capsules, granules, etc.) and can be applied percutaneously (for example, patches, etc.). Among them, tablets, fine granules and capsules are particularly preferred.

These preparations of the present invention can be produced by a per se known method (for example, method described in Japanese Pharmacopoeia, etc.) or an analogous method thereto, and a conventionally used pharmacologically acceptable carrier is appropriately used in appropriate amount.

The pharmaceutical composition for preventing or treating sleep disorder of the present invention can be used effectively for preventing and/or treating sleep disorder (insomnia) of a mammal (e.g., human, cat, dog, monkey, etc.). Examples of such sleep disorder include:
(1) dyssomnia such as intrinsic sleep disorders (e.g., psychophysiological insomnia), extrinsic sleep disorders, and circadian rhythm disorders (e.g., time zone change syndrome (jet lag), shift-work sleep disorder, irregular sleep wake pattern, delayed sleep-phase syndrome, advanced sleep-phase syndrome, non 24-hour sleep-wake disorder);
(2) parasomnias;
(3) sleep disorders associated with medical/psychiatric disorders (e.g., chronic occlusive pulmonary disease, Alzheimer's disease, Parkinson's disease, multiinfarct dementia, schizophrenia, depression, anxiety disorders).

In addition, in the present specification, the sleep disorder (insomnia) includes difficulty falling asleep (sleep-onset insomnia), arousals and awakenings during sleep (sleep maintenance insominia), early-morning awakening, and a combination thereof.

In addition, since natural sleep is induced, sleep latency is shortened, deep sleep is increased, maintenance of sleep is excellent and appropriate sleep duration can be attained by using the pharmaceutical composition for preventing or treatingsleep disorder of the present invention, nighttime awakenings can be reduced. Furthermore, the incidence of bruise and fracture can be reduced because nighttime awakening is reduced and also, just in case nighttime awakening happened, falling accident at the time of standing up is hard to happen.

In addition, the pharmaceutical composition for preventing or treating sleep disorder of the present invention can maintain the sleep-inducing effect in a long-term administration.

Furthermore, in the pharmaceutical composition for preventing or treating sleep disorder of the present invention, rebound phenomenon after cessation of administration is inhibited.

Moreover, pharmaceutical compositions comprising a combination of one or more drugs selected from the group consisting of sedative antidepressants and antihistamines, and (S)—N-[2-(1,6,7,8-tetrahydro-2H-indeno[5,4-b]furan-8-yl)ethyl]propionamide are effective for preventing and/or treating seasonal depression, genital and neuroendocrine disease, senile dementia, Alzheimer's disease, aging-related various disorder (e.g., prevention of aging, etc.), cerebral circulation disorder (apoplexy, etc.), head trauma, myelopathy, stress, epilepsy, convulsion, anxiety, depression, Parkinson's disease, hypertension, glaucoma, cancer, diabetes, headache, nocturnal pollakiuria, irritable bowel syndrome, and the like in addition to sleep disorder, and also for immunomodulation, intellectual tropism, mental stability and ovulation adjustment (e.g., contraception).

Furthermore, compound A can be effectively used alone, that is, without combining with sedative antidepressants and antihistamines, for preventing and/or treating sleep disorder (insomnia) of a mammal (e.g., human, cat, dog, monkey, etc.). Examples of such sleep disorder include:
(1) dyssomnia such as intrinsic sleep disorders (e.g., psychophysiological insomnia), extrinsic sleep disorders, and circadian rhythm disorders (e.g., time zone change syndrome (jet lag), shift-work sleep disorder, irregular sleep wake pattern, delayed sleep-phase syndrome, advanced sleep-phase syndrome, non 24-hour sleep-wake disorder);
(2) parasomnias;
(3) sleep disorders associated with medical/psychiatric disorders (e.g., chronic occlusive pulmonary disease, Alzheimer's disease, Parkinson's disease, multiinfarct dementia, schizophrenia, depression, anxiety disorders).

In addition to these, compound A can be used effectively for antiemesis and the like.

Further, for the purpose of obtaining a more highly qualified sleep in the prevention or treatment of these sleep disorders (insomnia), compound A can be suitably used in combination with a serotonin 2A antagonist (e.g., M-100907, ACP-103, APD-125, Org-50081, Mirtazapine (Org-4420 (s-miltazapine)), Pruvanserin, Eplivanserin, Quetiapine, Clozapine, Risperidone, Sertindole, Olanzapine, Ziprasidone, Asenapine, Ocaperidone, Paliperidone, R-167154, Iloperidone, Aripiprazole, desmethylclozapine (ACP-104, N-desmethylclozapine)). As in the case of the present invention mentioned above, the embodiment may be, for example, (1) a single preparation wherein a serotonin 2A antagonist and compound A are formulated with an appropriate pharmaceutically acceptable excipient etc., if desired, according to a known production method for formulating pharmaceutical preparations, (2) preparations wherein each of the active ingredients is formulated with an appropriate pharmaceutically acceptable excipient etc., if desired, so as to be used in combination (combined use) simultaneously or at different times, or (3) a combined set (kit products etc.) of preparations wherein each of the active ingredients is separately formulated appropriately with an excipient by a conventional method.

The above-mentioned serotonin 2A antagonist is a known compound, and available as commercial products, or can be produced according to methods known in the art.

M-100907 can be produced according to a method described in, for example, Medicinal Chemistry Research, 1996, 6, pp.1-10, or analogous method thereto.

ACP-103 can be produced according to a method described in, for example, WO 2001/66521, or analogous method thereto.

APD-125 can be produced according to a method described in, for example, WO 2005/12254, or analogous method thereto. org-50081 can be produced according to a method described in, for example, EP 0373998, or analogous method thereto.

Org-4420 can be produced according to a method described in, for example, U.S. Pat. No. 4,062,848, or analogous method thereto.

Pruvanserin can be produced according to a method described in, for example, WO 2001/07435, or analogous method thereto.

Eplivanserin can be produced according to methods described in, for example, WO 2005/05410, or analogous method thereto.

Asenapine can be produced according to a method described in, for example, U.S. Pat. No. 4,145,434, or analogous method thereto.

Ocaperidone can be produced according to a method described in, for example, EP 4453042A, or analogous method thereto.

Paliperidone can be produced according to a method described in, for example, U.S. Pat. No. 5,158,952, or analogous method thereto.

R-167154 can be produced according to a method described in, for example, WO 97/38991 and WO 99/19317, or analogous methods thereto.

Iloperidone can be produced according to a method described in, for example, WO 93/09102, or analogous method thereto.

Desmethylclozapine (ACP-104, N-desmethylclozapine) can be produced according to a method described in, for example, WO 2004/064753, or analogous method thereto.

The pharmaceutical composition for preventing or treating sleep disorder of the present invention is low toxic, and can be safely administered orally to a mammal such as human. The pharmaceutical composition for preventing or treating sleep disorder of the present invention can be administered before bedtime, at the moment of waking during the night, and/or at the moment of waking early in the morning depending on the kind of the above-mentioned sleep disorder (insomnia). Furthermore in the same way, when compound A is used alone, the pharmaceutical composition containing compound A can be administered before bedtime, at the moment of waking during the night, and/or at the moment of waking early in the morning depending on the kind of the above-mentioned sleep disorder (insomnia).

The dosage of the pharmaceutical composition for preventing or treating sleep disorder of the present invention is varied depending on the subject to be administered, route of administration, disease, kind of active ingredients to be used, and the like. For example, the following amount in terms of dosage of each of active ingredients may be administered per day in a single dose or in divided doses (preferably, administration of once a day) to an adult (body weight about 60kg) with sleep disorder, and it is preferred that each ingredient is administered in combination simultaneously or at different times of from 30 minutes to 3 hours.

When the pharmaceutical composition for preventing or treating for sleep disorder of the present invention comprising a combination of one or more drugs selected from the group consisting of sedative antidepressants and antihistamines, and compound A is used, the dosage for them may be reduced compared to the case when the drugs selected from the group consisting of sedative antidepressants and antihistamines, and compound A are used alone, respectively.

For example, in the case of a pharmaceutical composition comprising a combination of one kind of drugs selected from the group consisting of sedative antidepressants and antihistamines, and compound A, the dose of compound A in single daily administration is about 0.05 mg to about 50 mg, in particular in case of an oral preparation, preferably about 1 mg to about 20 mg as a single dose, and in case of a transdermal preparation, preferably about 0.1 mg to about 10 mg as a single dose.

In addition, the dose of one kind of the drug selected from the group consisting of sedative antidepressants and antihistamines may be suitably selected depending on the kind of the drug, and for example, in a single daily administration, it is usually about 0.1 mg to about 3,000 mg, and preferably about 5 mg to about 900 mg as a single dose.

The dose of doxepin as a more specific compound is, for example, in a single daily administration, about 0.1 mg to about 200 mg, preferably about 0.5 mg to about 30 mg, more preferably about 1 mg to about 20 mg, and particularly preferably about 1 mg to about 10 mg as a single dose.

Combination ratio (administration ratio) of one kind of the drug selected from the group consisting of sedative antidepressants and antihistamines with compound A in the pharmaceutical composition for preventing or treating sleep disorder of the present invention is usually 1:100 to 60:1 (weight ratio), preferably 1:20 to 30:1 (weight ratio), more preferably 1:10 to 30:1 (weight ratio), and particularly preferably 1:10 to 20:1 (weight ratio) As for a more specific compound, the combination ratio (administration ratio) of doxepin and compound A is, for example, 1:50 to 30:1 (weight ratio), preferably 1:20 to 10:1 (weight ratio), more preferably 1:10 to 5:1 (weight ratio).

When two or more kinds of drugs selected from the group consisting of sedative antidepressants and antihistamines are used, each dosage of the drugs can be reduced compared to when only one of them is used.

Furthermore, the pharmaceutical composition for preventing or treating sleep disorder of the present invention may be jointly used in combination with other active ingredients as long as its advantageous property is substantially not interfered. The other active ingredients, sedative antidepressants and/or antihistamines and compound A may be blended according to a per se known method to give a pharmaceutical composition (e.g., tablets, powders, granules, capsules (including soft capsules), liquids, patches, injections, suppositories, sustained-release preparations, etc.), and the obtained pharmaceutical composition may be administered, or preparations formulated separately may be administered to the same subject simultaneously or at different times in the same way of the preparation of the present invention.

The present invention will be described in detail through the following Experiments and Preparation Examples. However, these are just an example, and are not intended to limit the present invention, and may be changed without departing from the scope of the present invention.

### PREPARATION EXAMPLE 1

| | |
|---|---|
| (1) Doxepin | 8.0 g |
| (2) Compound A | 8.0 g |
| (3) Lactose | 60.0 g |
| (4) Corn starch | 35.0 g |
| (5) Gelatin | 3.0 g |
| (6) Magnesium stearate | 2.0 g |

A mixture of doxepin 8.0 g, compound A 8.0 g, lactose 60.0 g and corn starch 35.0 g was granulated through 1 mm mesh sieve using 30 ml of 10% by weight aqueous solution of gelatin (3.0 g as gelatin), and the granules were dried at 40° C., and passed through the sieve again. The resulting granules are mixed with magnesium stearate 2.0 g, and the mixture is compressed. The resulting core tablets are sugar-coated using a suspension of sucrose, titanium oxide, talc and gum arabic in water. The coated tablets are burnished with yellow beeswax to give 1,000 coated tablets.

Experiment 1

As test animals, cats raised under the circumstance of 12-hour light-dark cycle (light period from 7 a.m. to 7 p.m.) were used. Under pentobarbital anesthesia, electrodes for recording electroencephalogram were implanted in frontal lobe of cerebral cortex, frontal lobe and hippocampus, respectively, and electrode for electrooculogram was implanted in orbit bone, and stainless wire electrode for recording electromyogram was implanted in dorsal cervical muscle. An antibiotic was administered to prevent bacterial infection. Taming to cage for measuring electroencephalogram was started from 3 to 4 days after the operation, and measurement of electroencephalogram was carried out after 1 to 2 weeks of taming.

Vehicle and drugs to be administered were filled in a capsule, and it was compulsorily administered orally. Treated groups were comprised of 10 animals per one group, and crossover test was carried out.

[Drug Administration Group]

(1) Vehicle (0.5% aqueous methylcellulose solution)
(2) Compound A (0.1 mg/kg)
(3) Doxepin (0.3 mg/kg)
(4) Combined use (compound A (0.1 mg/kg)+doxepin (0.3 mg/kg))

After being attached the electrodes for measurement from about 8:30 a.m., the cats were placed in test box. Administration was conducted at around 10:00 a.m., and the sleep electroencephalogram for 8 hours after administration was measured.

Electroencephalogram data was obtained using Synafit 2500 of NEC Saneisha. Sleep Sign Ver. 2.0 that is a program for sleep analysis study of Kissei Comtec Co. LTD., was used for analysis. As characteristic parameter, δ wave was set by the waveform derived from cortex and θ wave was set by the waveform derived from hippocampus. Analysis was carried out every 20 seconds with If . . . Then method below using this parameter, and Wakefulness, Slow-Wave Sleep and REM Sleep were automatically judged. Furthermore, after completion of these automatic judgments, visual judgment was conducted and the judgment was modified when the judgment of sleep stage was apparently different.

[Automatic Judgment of Sleep Electroencephalogram]

a<EMG Integral<100→Yes “Wakefulness”

↓No
20<Delta % Time<100 & 0<EMG Integral<b →Yes “SWS”

↓No
25<Theta % Time<100 & 0<EMG Integral<c →Yes “REM”

↓No
3<fast wave % Time<100 →Yes “Wakefulness”

↓No

Previous Stage (reflect immediately preceding judgment of sleep)
* values of a, b, c should be determined with an appropriate numerical value for each body.

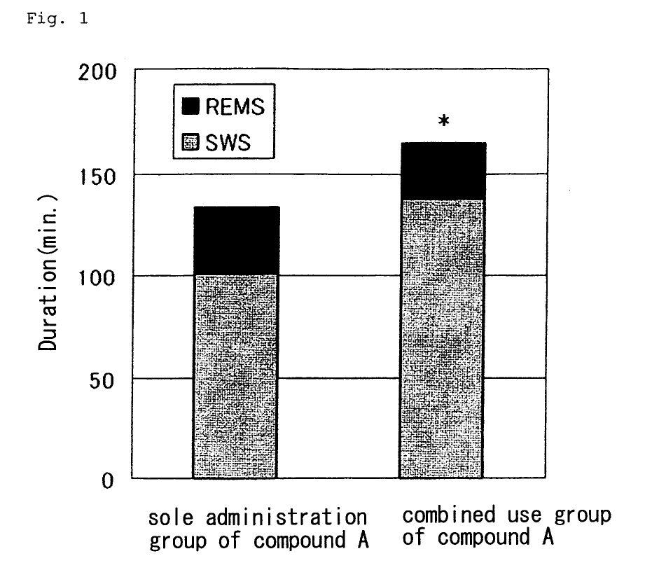

The results for the sole administration group of compound A and the combined use group of compound A and doxepin are shown in FIG. 1. As is evident from this Figure, in the combined use group of compound A and doxepin, as compared with the sole administration group of compound A, the total slow-wave sleep time and the total sleep duration were prolonged by about 30 minutes respectively, and a significant difference was observed.

Experiment 2

[Frequency Analysis]

In Experiment 1, using Sleep Sign Ver. 2.0 (KISSEI COMTEC), only the epochs that were judged as slow-wave sleep was extracted, and subjected to FFT analysis, and power spectrum value in each frequency was determined.

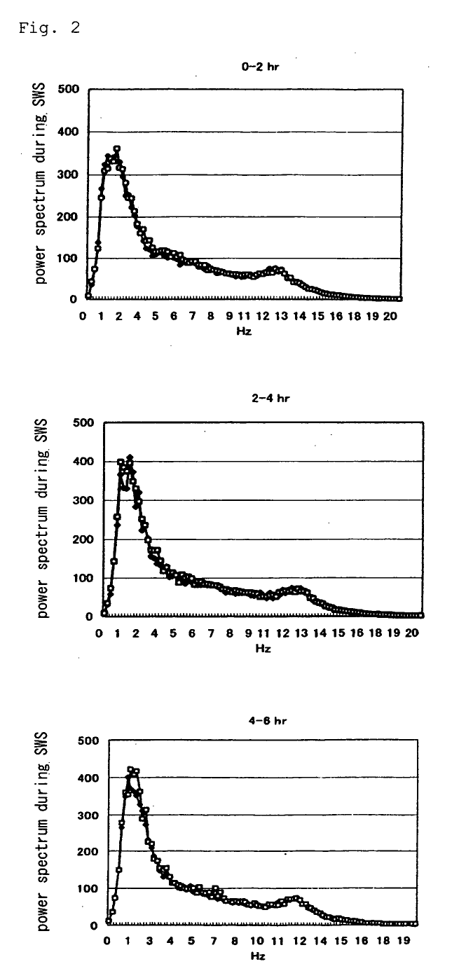

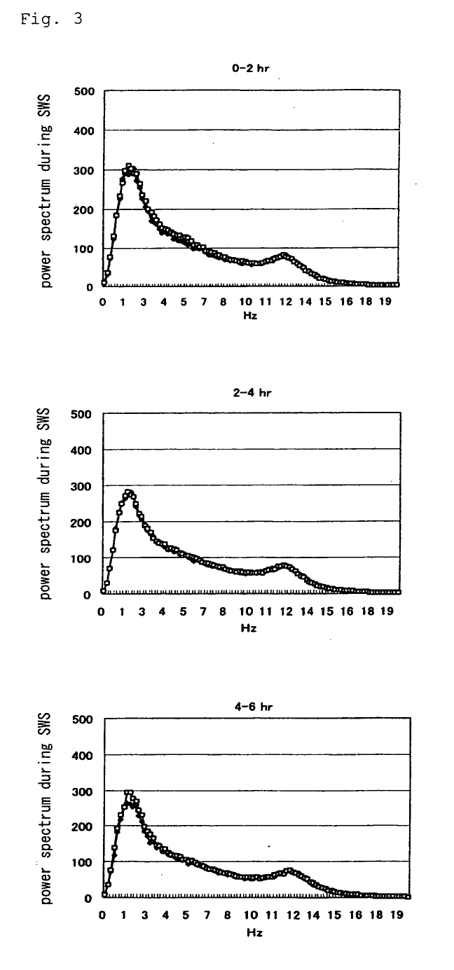

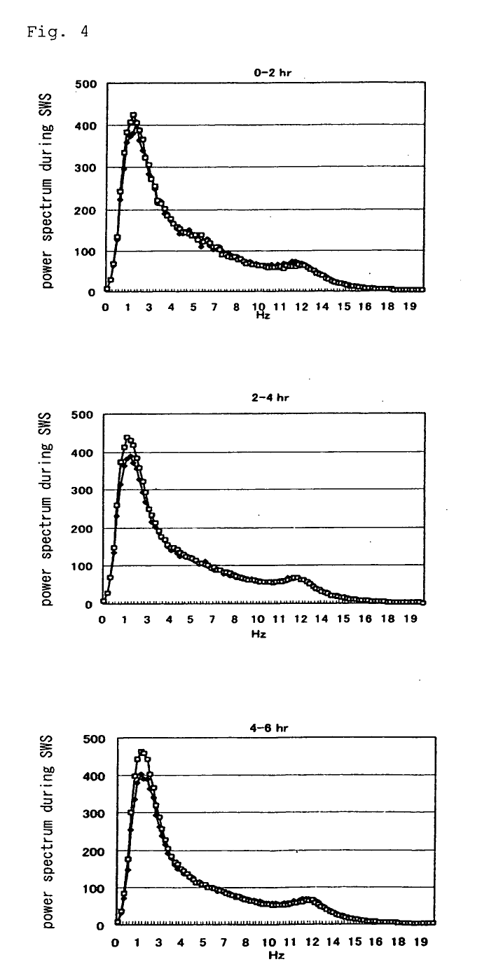

The results are shown in FIG. 2 (in the figure, ♦ vehicle and □ compound A), in FIG. 3 (in the figure, ♦ vehicle and □ doxepin) and in FIG. 4 (in the figure, ♦ compound A and □ combined use of compound A and doxepin) (0 to 2 hr, 2 to 4 hr, and 4 to 6 hr, respectively after the administration). As is evident from these Figures, as compared with the sole administration group of compound A, an increase of power spectrum in the low-frequency region (in the vicinity of 2 Hz) was observed from about 2 hours after the administration in the combined use group of compound A and doxepin. From this result, it was found out that the sleep becomes deeper in the combined use group of compound A and doxepin.

### INDUSTRIAL APPLICABILITY

According to the present invention, there is provided a pharmaceutical composition for preventing or treating sleep disorder, which induces natural sleep, shortens sleep latency, increases deep sleep, is excellent in maintaining sleep, and exerts to attain appropriate sleep duration.

In addition, according to the present invention, there is provided a pharmaceutical composition for preventing or treating sleep disorder wherein the sleep-inducing effect is maintained in a long-term administration.

Furthermore, according to the present invention, there is provided a pharmaceutical composition for preventing or treating sleep disorder wherein the rebound after cessation of administration is inhibited.

1. A pharmaceutical composition for preventing or treating sleep disorder, which comprises (S)—N-[2-(1,6,7,8-tetrahydro-2H-indeno[5,4-b]furan-8-yl)ethyl]propionamide in combination with one or more drugs selected from the group consisting of sedative antidepressants and antihistamines.

2. A pharmaceutical composition for preventing or treating sleep disorder, which comprises (S)—N-[2-(1,6,7,8-tetrahydro-2H-indeno[5,4-b]furan-8-yl)ethyl]propionamide in combination with doxepin.

3. Use of (S)—N-[2-(1,6,7,8-tetrahydro-2H-indeno[5,4-b]furan-8-yl)ethyl]propionamide in combination with one or more drugs selected from the group consisting of sedative antidepressants and antihistamines, for the manufacture of a pharmaceutical composition for preventing or treating sleep disorder.

4. Use of (S)—N-[2-(1,6,7,8-tetrahydro-2H-indeno[5,4-b]furan-8-yl)ethyl]propionamide in combination with doxepin, for the manufacture of a pharmaceutical composition for preventing or treating sleep disorder.

5. A method for preventing or treating sleep disorder, which comprises administering (S)—N-[2-(1,6,7,8-tetrahydro-2H-indeno[5,4-b]furan-8-yl)ethyl]propionamide in combination with one or more drugs selected from the group consisting of sedative antidepressants and antihistamines to a subject.

6. A method for preventing or treating sleep disorder, which comprises administering (S)—N-[2-(1,6,7,8-tetrahydro-2H-indeno[5,4-b]furan-8-yl)ethyl]propionamide in combination with doxepin to a subject.
